SEQUENCE LISTING (6 sequences)

SEQ ID NO: 1 - protein, 4 aa, Artificial Sequence
  Peptide fragment
CSLY

SEQ ID NO: 2 - protein, 4 aa, Artificial Sequence
  Peptide fragment
YQLE

SEQ ID NO: 3 - protein, 3 aa, Artificial Sequence
  Peptide fragment
CSL

SEQ ID NO: 4 - protein, 3 aa, Artificial Sequence
  Peptide fragment
SLY

SEQ ID NO: 5 - protein, 3 aa, Artificial Sequence
  Peptide fragment
YQL

SEQ ID NO: 6 - protein, 3 aa, Artificial Sequence
  Peptide fragment
QLE

(19) United States
(12) Patent Application Publication
(10) Pub. No.: US 2019/0389903 A1
(43) Pub. Date: Dec. 26, 2019

(54) INSULIN A-CHAIN DERIVED PEPTIDE FRAGMENT AND PHARMACEUTICAL COMPOSITION FOR PREVENTING OR TREATING DIABETES OR DIABETIC WOUNDS, CONTAINING THE SAME

(71) Applicant: SUPADELIXIR INC., Chuncheon-si (KR)

(72) Inventors: Jang-Hee HAHN, Chuncheon-si (KR); Chang-Gyum KIM, Chuncheon-si (KR)

(21) Appl. No.: 16/484,026

(22) PCT Filed: Feb. 7, 2018
(86) PCT No.: PCT/KR2018/001620

§ 371 Date: Aug. 6, 2019

(30) Foreign Application Priority Data
Feb. 9, 2017 (KR) 10-2017-0018116

(51) Int. Cl. C07K 5/103; C07K 5/087; C07K 5/107; C07K 5/083

(52) U.S. Cl. CPC C07K 5/1013; C07K 5/0812; A61K 38/00; C07K 5/0808; C07K 5/1016

(57) ABSTRACT

The present invention relates to an insulin-A-chain-derived peptide fragment and a pharmaceutical composition for the prevention or treatment of diabetes or diabetic wounds, containing the same as an active ingredient. The peptide of the present invention induces endocytosis through an insulin receptor and exhibits activity of enhancing glucose uptake into cells by translocating a glucose transporter to a cell membrane, and of increasing cell migration and proliferation. Therefore, the peptide of the present invention can be efficiently applied to the prevention or treatment of diabetes or diabetic wounds.

### CROSS-REFERENCE TO RELATED APPLICATIONS

This application claims the benefit under 35 U.S.C. section 371, of PCT International Application No.: PCT/KR2018/001620, filed on Feb. 7, 2018, which claims foreign priority to Korean Patent Application No.: KR10-2017-0018116, filed on Feb. 9, 2017, in the Korean Intellectual Property Office, both of which are hereby incorporated by reference in their entireties.

### TECHNICAL FIELD

The present invention relates to an insulin-A-chain-derived peptide fragment and a pharmaceutical composition for preventing or treating diabetes or diabetic wounds containing the same as an active ingredient.

### BACKGROUND ART

Insulin is a peptide hormone produced by beta cells of pancreatic islets. Insulin regulates the metabolism of carbohydrates, fats and proteins by promoting the uptake of glucose from the blood into the fat, liver, and skeletal muscle cells. The human insulin protein is composed of 51 amino acids and has a molecular weight of 5808 Da. Insulin is a dimer of A- and B-chains, which are linked together by disulfide bonds.

Diabetes is a metabolic disease characterized by high blood glucose levels caused by decreased insulin secretion, reduced use of glucose, or increased production of glucose. Diabetes causes a variety of complications, for example, diabetic wounds such as diabetic foot ulcers, etc. Currently, human insulin preparations or recombinant insulin preparations are clinically used for the treatment of diabetes requiring insulin therapy. Examples of the recombinant insulin preparation include Humulin R™ injections and Humulin N™ injections.

Meanwhile, the conventional insulin preparations, which are high-molecular-weight protein preparations, are used in the form of injections and are inconvenient to use, and care must also be taken to store and handle the preparations. Moreover, it takes a long time to produce high-purity insulin, and high costs are incurred in the production and purification processes.

### DISCLOSURE

### Technical Problem

The present inventors have designed various peptide fragments having three amino acids (trimers) or four amino acids (tetramers) from insulin and evaluated the activity thereof. Surprisingly, the present inventors have found that specific peptide fragments having three or four amino acids derived from the 11^{th} to 17^{th} amino acid sequence of the insulin A-chain have insulin-like activity. In particular, the present inventors have ascertained that the above specific peptide fragment induces endocytosis of the insulin receptor, enhances the glucose uptake into cells by translocating a glucose transporter to a cell membrane, and increases cell migration and proliferation.

Accordingly, an objective of the present invention is to provide an insulin-A-chain-derived specific peptide fragment.

Another objective of the present invention is to provide a pharmaceutical composition for the prevention or treatment of diabetes or diabetic wounds, containing the insulin-A-chain-derived specific peptide fragment as an active ingredient.

### Technical Solution

An aspect of the present invention provides a peptide comprising three or four amino acids represented by Chemical Formula 1 below:

X-A-Leu-Y <Chemical Formula 1>

in which A is serine (Ser) or glutamine (Gln); X is hydrogen, tyrosine (Tyr) or cysteine (Cys); Y is hydrogen, tyrosine (Tyr) or glutamic acid (Glu); neither X nor Y is hydrogen; and — is a peptide bond.

In an embodiment, the peptide of the present invention may be a peptide represented by Chemical Formula 1a below:

X1-Ser-Leu-Y1 <Chemical Formula 1a>

in which X1 is hydrogen or cysteine (Cys); Y1 is hydrogen or tyrosine (Tyr); neither X1 nor Y1 is hydrogen; and — is a peptide bond. In this embodiment, the peptide of the present invention may be a peptide selected from the group consisting of SEQ ID NOS: 1, 3, and 4, and is preferably a peptide of SEQ ID NO: 3.

In another embodiment, the peptide of the present invention may be a peptide represented by Chemical Formula 1b below:

X2-Gln-Leu-Y2 <Chemical Formula 1b>

in which X2 is hydrogen or tyrosine (Tyr); Y2 is hydrogen or glutamic acid (Glu); neither X2 nor Y2 is hydrogen; and — is a peptide bond. In this embodiment, the peptide of the present invention may be a peptide selected from the group consisting of SEQ ID NOS: 2, 5, and 6.

Another aspect of the present invention provides a pharmaceutical composition for the prevention or treatment of diabetes or diabetic wounds, containing, as an active ingredient, the peptide comprising three or four amino acids as above.

In the pharmaceutical composition of the present invention, the peptide may be a peptide selected from the group consisting of SEQ ID NOS: 1 to 6, and is preferably a peptide of SEQ ID NO: 3. In an embodiment, the pharmaceutical composition of the present invention may be provided in the form of a transdermal delivery system.

### Advantageous Effects

According to the present invention, insulin-A-chain-derived specific peptide fragments (peptide fragments or small peptides) are proven to have insulin-like activity. In particular, according to the present invention, it is confirmed that the specific peptide fragment is capable of inducing endocytosis through an insulin receptor, enhancing the glucose uptake into cells by translocating a glucose transporter to a cell membrane, and increasing cell migration and proliferation. Thus, the peptide of the present invention can be efficiently applied to the prevention or treatment of diabetes and diabetic wounds. Also, the peptide of the present invention is a small molecule of 500 Da or less, and can thus be provided not only in the form of an injection but also in the form of a transdermal delivery system. Furthermore, since the peptide of the present invention is a stable molecule formed through covalent bonding, it can be stored without being influenced by temperature, and the storage period thereof is very long, so it is easy to handle and store. In addition, the peptide of the present invention can be manufactured in a simple manner at low cost compared to conventional insulin preparations.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the results of evaluation of insulin-like activity by measuring a physical interaction between Grb2 and SOS1 through PLA (proximity ligation assay) after treatment of cells with the peptide of the present invention;

FIG. 2 shows the results of immunofluorescence (FIG. 2A) and the results of RNA interference (FIG. 2B) in order to evaluate whether the peptide of the present invention induces endocytosis by binding to an insulin receptor;

FIGS. 3 and 4 show the results of testing of cell migration (FIG. 3) and cell proliferation (FIG. 4) in order to evaluate whether the peptide of the present invention promotes cell migration and proliferation;

FIG. 5 shows the results of analysis of 2-NBDG glucose uptake in order to evaluate the activity of the peptide of the present invention on glucose uptake;

FIG. 6 shows the results of testing of 2-NBDG glucose uptake after inhibition of insulin receptor expression using siRNA in order to evaluate whether the peptide of the present invention enhances glucose uptake through the insulin receptor; and

FIG. 7 shows the results of evaluation of translocation of the glucose transporter to a cell membrane in HaCaT cells through immunofluorescence.

### MODE FOR INVENTION

The present invention pertains to a peptide having three amino acids (trimer) or four amino acids (tetramer) manufactured from the 11^{th} to 17^{th} amino acid sequence (i.e. Cys-Ser-Leu-Tyr-Gln-Leu-Glu) of an insulin A-chain, and particularly to a peptide comprising three or four amino acids represented by Chemical Formula 1 below:

X-A-Leu-Y <Chemical Formula 1>

in which A is serine (Ser) or glutamine (Gln); X is hydrogen, tyrosine (Tyr) or cysteine (Cys); Y is hydrogen, tyrosine (Tyr) or glutamic acid (Glu); neither X nor Y is hydrogen; and — is a peptide bond.

The peptide in which X is hydrogen in Chemical Formula 1 is a peptide in which a peptide bond is absent at the corresponding position, and thus, A, that is, serine (Ser) or glutamine (Gln), is an N-terminal amino acid of the corresponding peptide. Also, the peptide in which Y is hydrogen is a peptide in which a peptide bond is absent at the corresponding position, and thus, leucine (Leu) is a C-terminal amino acid of the corresponding peptide.

The peptide of the present invention has insulin-like activity, and in particular induces endocytosis through the insulin receptor and enhances the glucose uptake into cells by translocating the glucose transporter to the cell membrane, and moreover increases cell migration and proliferation.

In an embodiment, the peptide of the present invention may be a peptide represented by Chemical Formula 1a below:

X1-Ser-Leu-Y1 <Chemical Formula 1a>

in which X1 is hydrogen or cysteine (Cys); Y1 is hydrogen or tyrosine (Tyr); neither X1 nor Y1 is hydrogen; and — is a peptide bond.

The peptide in which X1 is hydrogen in Chemical Formula 1a is a peptide in which a peptide bond is absent at the corresponding position, and thus, serine (Ser) is an N-terminal amino acid of the corresponding peptide. Also, the peptide in which Y1 is hydrogen is a peptide in which a peptide bond is absent at the corresponding position, and thus, leucine (Leu) is a C-terminal amino acid of the corresponding peptide.

The peptide represented by Chemical Formula 1a is preferably a peptide selected from the group consisting of SEQ ID NOS: 1, 3, and 4, and is more preferably a peptide of SEQ ID NO: 3.

In another embodiment, the peptide of the present invention may be a peptide represented by Chemical Formula 1b below:

X2-Gln-Leu-Y2 <Chemical Formula 1b>

in which X2 is hydrogen or tyrosine (Tyr); Y2 is hydrogen or glutamic acid (Glu); neither X2 nor Y2 is hydrogen; and — is a peptide bond.

The peptide in which X2 is hydrogen in Chemical Formula 1b is a peptide in which a peptide bond is absent at the corresponding position, and thus, glutamine (Gln) is an N-terminal amino acid of the corresponding peptide. Also, the peptide in which Y2 is hydrogen is a peptide in which a peptide bond is absent at the corresponding position, and thus, leucine (Leu) is a C-terminal amino acid of the corresponding peptide.

The peptide represented by Chemical Formula 1b is preferably a peptide selected from the group consisting of SEQ ID NOS: 2, 5, and 6.

In addition, the present invention pertains to a pharmaceutical composition for the prevention or treatment of diabetes or diabetic wounds, containing, as an active ingredient, the peptide comprising three or four amino acids represented by Chemical Formula 1. The diabetic wounds may be typically exemplified by diabetic foot ulcers.

In the pharmaceutical composition of the present invention, the peptide may be a peptide comprising three or four amino acids represented by Chemical Formula 1a or 1b, as described above. Also, in the pharmaceutical composition of the present invention, the peptide may be a peptide selected from the group consisting of SEQ ID NOS: 1 to 6, and is preferably a peptide of SEQ ID NO: 3.

The pharmaceutical composition of the present invention may include a pharmaceutically acceptable carrier, examples of which include excipients such as lactose, corn starch, etc., lubricants such as magnesium stearate, etc., emulsifiers, suspension agents, buffering agents, isotonic agents, etc., which are known in the art. The pharmaceutical composition of the present invention may be formulated in an oral dosage form or a parenteral dosage form, and is preferably provided in a dosage form such as a transdermal delivery system. For example, as for intramuscular, intraperitoneal, subcutaneous and intravenous administration forms, the pharmaceutical composition may include a buffering agent that is able to prepare a sterile solution of the active ingredient and to appropriately adjust the pH of the solution. For intravenous administration, an isotonic agent may be included so as to impart isotonicity to the formulation. Also, the pharmaceutical composition of the present invention may be provided in the form of an aqueous solution including a pharmaceutically acceptable carrier, such as saline having a pH of 7.4, and may be topically injected in the form of a solution into the intramuscular blood stream of the patient. Furthermore, examples of the transdermal delivery system include a solution for external use, an emulsion, an ointment, a patch, etc., which may be formulated in accordance with a typical pharmaceutical method. The pharmaceutical composition of the present invention may be administered to patients exhibiting diabetes and/or diabetic wounds at a dose of about 1 to 10 mg/kg/day. Appropriate doses may typically vary depending on the patient's age, weight and symptoms.

A better understanding of the present invention will be given through the following examples and test examples, which are merely set forth to illustrate the present invention but are not to be construed as limiting the scope of the present invention.

### Example 1. Synthesis of Peptide

Peptides of SEQ ID NOS: 1 to 6 (Table 1 below) were synthesized through an FMOC solid-phase method using an automated synthesizer (PeptrEx-R48, Peptron Corporation, Daejeon, Korea). The synthesized peptides were purified and analyzed through reverse-phase high-performance liquid chromatography (HPLC) (Prominence LC-20AB, Shimadzu, Japan) using a C18 analysis RP column (Shiseido Capcell Pak), and identified using a mass spectrometer (HP 1100 Series LC/MSD, Hewlett-Packard, Roseville, USA).

TABLE 1

| Peptide name | SEQ ID NO: | Amino acid sequence |
|---|---|---|
| IDP1 | SEQ ID NO: 1 | Cys-Ser-Leu-Tyr |
| IDP2 | SEQ ID NO: 2 | Tyr-Gln-Leu-Glu |
| IDP3 | SEQ ID NO: 3 | Cys-Ser-Leu |
| IDP4 | SEQ ID NO: 4 | Ser-Leu-Tyr |
| IDP5 | SEQ ID NO: 5 | Tyr-Gln-Leu |
| IDP6 | SEQ ID NO: 6 | Gln-Leu-Glu |

### Example 2. Preparation of Composition Containing Peptide

Each of the peptides of SEQ ID NOS: 1 to 6 was dissolved in PBS to give a 1 M solution. The protein solution thus obtained was used for the following test examples.

### Test Example 1. Evaluation of Insulin-Like Activity

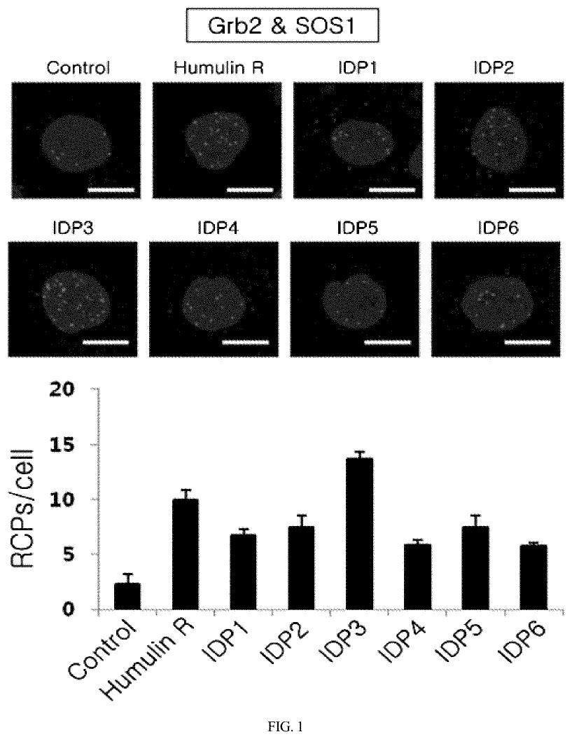

When the insulin signal is transmitted into cells, the physical interaction between Grb2 and SOS1 is increased. After treatment of the cells with the peptide of the present invention, the physical interaction between Grb2 and SOS1 was measured through PLA (proximity ligation assay) and thus insulin-like activity was evaluated. 4.5×10⁴ HaCaT cells (CLS 300493) were seeded in a DMEM serum medium in each well of a 24-well microplate and cultured in a 5% CO₂ incubator at 37° C. for 24 hr, after which the peptide solution of Example 2 was added such that the peptide concentration of SEQ ID NOS: 1 to 6 in the medium was 1 μM, followed by incubation for 5 min under the same conditions. As a positive control, Humulin R was treated at the same concentration. A control received no treatment. The cells of each well were washed with PBS, treated with 2% formaldehyde for 15 min to thus fix the same, and then treated with 0.1% TritonX-100 for 5 min to thus increase antibody permeability into the cells. A Grb2 antibody (Santa Cruz, Calif., USA) and a SOS1 antibody (Santa Cruz, Calif., USA) were added thereto, and a PLA probe was added in accordance with a manufacturer's protocol using an in-situ PLA kit (Sigma-Aldrich), followed by hybridization, ligation, amplification and mounting. The physical interactions of the Grb2 and SOS1 antibodies were quantified by measuring the PLA signals detected in individual cells using a confocal laser microscope (Olympus FluoView FW1000; Olympus, Tokyo, Japan). The results are shown in FIG. 1. As is apparent from the results of FIG. 1, the peptide of the present invention exhibited significantly increased antibody interactions compared to the untreated control, indicating that it had insulin-like activity. In particular, the group treated with the peptide of SEQ ID NO: 3 showed a significantly higher antibody interaction than the positive control treated with an insulin preparation, Humulin R.

### Test Example 2. Immunofluorescence Analysis

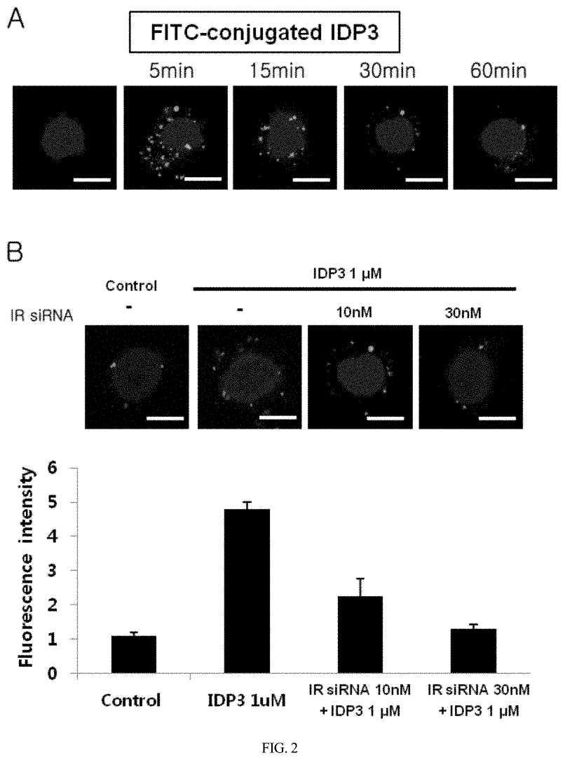

In order to evaluate whether, like insulin, the peptide of the present invention induces endocytosis by binding to the insulin receptor, immunofluorescence analysis was performed. A fluorescent material (FITC) was conjugated to the peptide of SEQ ID NO: 3. HaCaT cells (CLS 300493) were pretreated in the same manner as in Test Example 1, added with the peptide solution of SEQ ID NO: 3 having the fluorescent material conjugated thereto such that the final concentration was 1 μM, and then treated for 5 min, 15 min, 30 min, and 60 min. The cells were washed three times with a PBS solution and then mounted onto a slide to give a test specimen, which was then observed using a confocal laser microscope (Olympus FluoView FW1000; Olympus, Tokyo, Japan). The results are shown in FIG. 2A. As seen in FIG. 2A, the highest endocytosis was observed 5 min after treatment. The insulin binds to the insulin receptor in the cells, followed by endocytosis (internalization) and then degradation. Therefore, the above results show that the fluorescence appeared at the highest level 5 min after peptide treatment and then decreased due to degradation.

Based on the above test results, in order to determine whether the effect of the peptide of the present invention on endocytosis is mediated by the insulin receptor, RNA interference was analyzed. HaCaT cells (CLS 300493) were cultured in a DMEM serum medium until the cells reached 50-60% confluency, followed by transient transfection using the insulin receptor siRNA (Santa Cruz, Calif., USA) (no treatment, 10 nM treatment, or 30 nM treatment) and lipofectamine RNAiMAX (Invitrogen) in accordance with the manufacturer's protocol to thus inhibit the insulin receptor expression, after which the cells were treated for 5 min using the peptide of SEQ ID NO: 3 having the fluorescent material conjugated thereto at a concentration of 1 μM. The fluorescence intensity of each group was observed using a confocal laser microscope (Olympus FluoView FW1000; Olympus, Tokyo, Japan). The results are shown in FIG. 2B. As is apparent from the results of FIG. 2B, the incidence of endocytosis was reduced in the test group transiently transfected with the insulin receptor siRNA, indicating that the peptide of the present invention induced endocytosis through the insulin receptor, similarly to insulin.

### Test Example 3. Evaluation of Cell Migration and Proliferation Activity

In order to determine whether the peptide of the present invention promotes cell migration and proliferation, cell migration and proliferation tests were performed.

For cell migration testing, a Culture-Insert 2 Well (Ibidi™), having two separate culture spaces therein, was used. When cells are cultured on the above plate, the cells are attached to both sides, between which an empty space is formed, followed by removal of the insert well and further culturing, whereby the cells of the two sides migrate to the empty space of the plate. HaCaT cells (CLS 300493) were cultured on the above plate until the cells reached 90% confluency and then the insert well was removed, after which the cells were treated for 5 min with the peptide of SEQ ID NO: 3 (0.01 μM, 0.1 μM, 1 μM) or Humulin R (1 μM), and washed with PBS, after which the medium was replaced with a cell culture medium (DMEM serum medium). After 10 hr and 13 hr, cell migration was observed through a phase contrast microscope.

For cell proliferation testing, 5×10³ HaCaT cells (CLS 300493) were seeded in each well of a 96-well plate, and then treated with a solution containing the peptide of SEQ ID NO: 3 (total volume: 90 μl) such that the concentrations in serum-free media were 0.1, 1 and 10 μM. Cell proliferation for 24, 48 and 72 hr was observed using a microplate reader (Versa Max, USA). For measurement, treatment was performed for 2 hr using 10 μl of a CCK-8 (cell counting kit-8) solution at the same time every day.

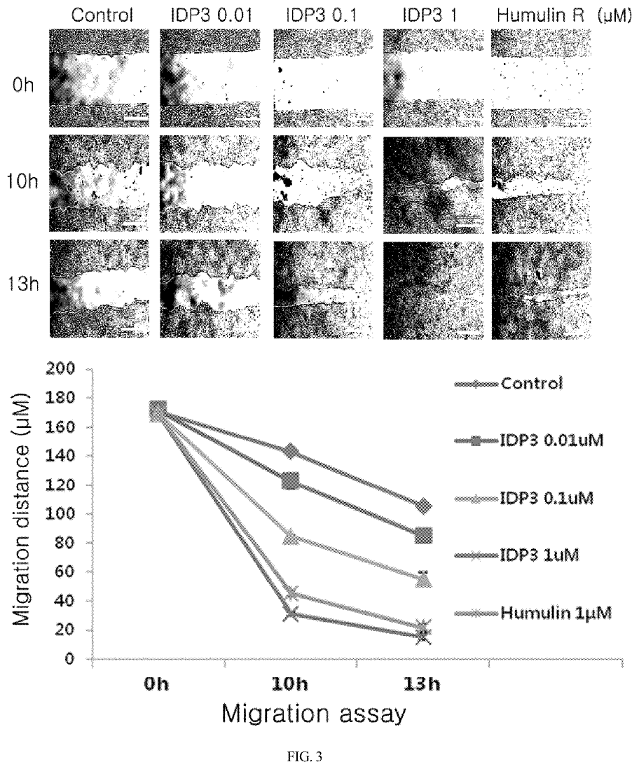

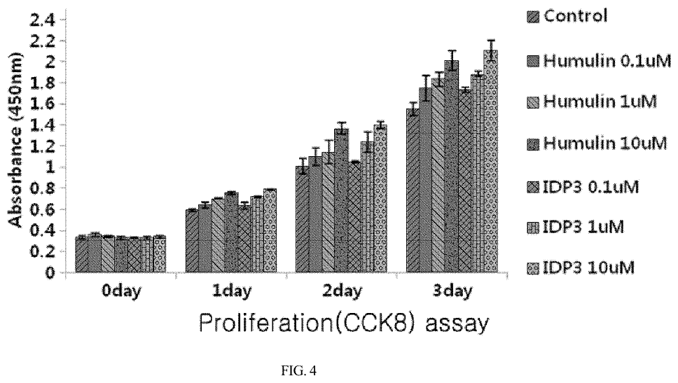

The cell migration and proliferation test results are shown in FIGS. 3 and 4, respectively. As is apparent from the results of FIGS. 3 and 4, it can be confirmed that cell migration and proliferation increased in a concentration-dependent manner, similarly to Humulin R used as the positive control, indicating that the peptide of the present invention was effective in the treatment of diabetic wounds.

### Test Example 4. Evaluation of Glucose Uptake Activity

A glucose transporter (GLUT4) translocates to the cell membrane by insulin stimulation to thus enhance the glucose uptake into cells. In order to evaluate whether the peptide of the present invention contributes to the glucose uptake, 2-NBDG glucose uptake analysis and immunofluorescence were performed.

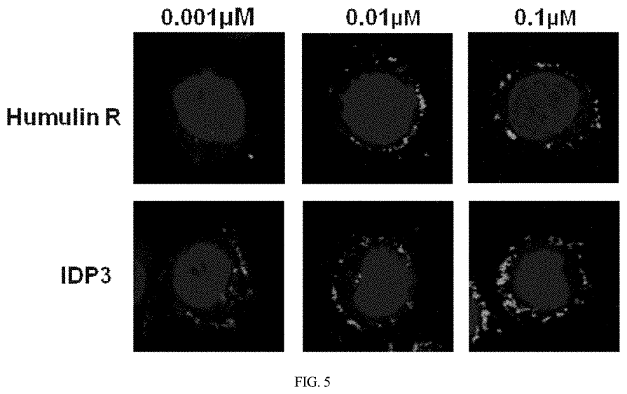

2-NBDG glucose, which is a fluorescent deoxyglucose analogue, is introduced into cells by a glucose transporter, like glucose, but accumulates without actual metabolism, and thus the extent of glucose uptake may be determined based on the accumulated amount of fluorescence. A 2-NBDG glucose uptake kit (Biovision) was used for 2-NBDG glucose uptake analysis. Specifically, 3×10⁴ HaCaT cells (CLS 300493) were seeded in each well of a 24-well plate, cultured in a 5% CO₂ incubator at 37° C. for 24 hr with a DMEM serum medium, washed with PBS, and treated with the peptide of SEQ ID NO: 3 and Humulin R such that the concentrations in serum-free media were 0.001 μM, 0.01 μM, and 0.1 μM, followed by incubation for 5 min. A mixed solution of a 2-NBDG reagent and a glucose uptake enhancer was prepared in accordance with the manufacturer's protocol, after which the cells were treated therewith for 1 hr, washed with the supplied cold 1× analysis buffer, and observed using a confocal laser microscope (Olympus FluoView FW1000; Olympus, Tokyo, Japan). The results are shown in FIG. 5. As is apparent from the results of FIG. 5, the glucose uptake was increased in a concentration-dependent manner, similarly to Humulin R used as the positive control, indicating that the peptide of the present invention enhanced glucose uptake into cells by translocating the glucose transporter to the cell membrane, similarly to insulin.

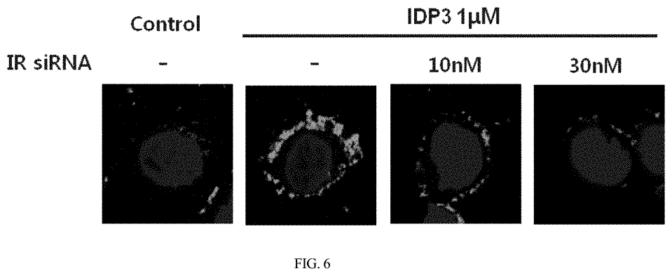

Based on the above test results, in order to evaluate whether, like insulin, the peptide of the present invention enhances glucose uptake through the insulin receptor, insulin receptor expression was inhibited using siRNA, after which 2-NBDG glucose uptake testing was performed. HaCaT cells (CLS 300493) were cultured in a DMEM serum medium until the cells reached 50-60% confluency, followed by transient transfection using the insulin receptor siRNA (Santa Cruz, Calif., USA) (no treatment, 10 nM treatment, or 30 nM treatment) and lipofectamine RNAiMAX (Invitrogen) in accordance with the manufacturer's protocol to thus inhibit insulin receptor expression, after which the cells were treated for 5 min using the peptide of SEQ ID NO: 3 at a concentration of 1 μM. A mixed solution of a 2-NBDG reagent and a glucose uptake enhancer was prepared in accordance with the manufacturer's protocol, after which the cells were treated therewith for 1 hr, washed with the supplied cold 1× analysis buffer, and observed using a confocal laser microscope (Olympus FluoView FW1000; Olympus, Tokyo, Japan). The results are shown in FIG. 6. As is apparent from the results of FIG. 6, the glucose uptake was reduced in the test group transiently transfected with the insulin receptor siRNA, indicating that the peptide of the present invention had an influence on the glucose uptake through the insulin receptor, similarly to insulin.

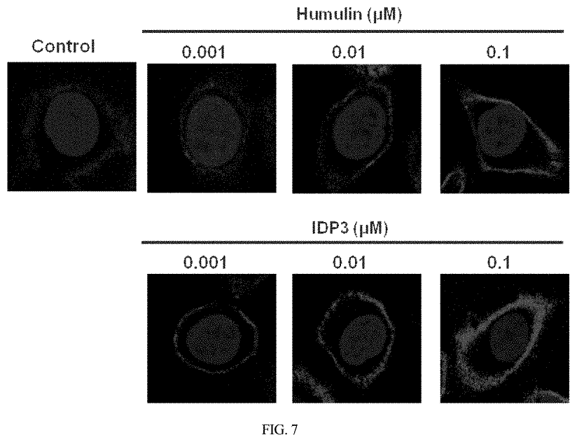

In addition, the translocation of the glucose transporter to the cell membrane in HaCaT cells was evaluated through immunofluorescence analysis. Specifically, HaCaT cells (CLS 300493) were treated with the peptide of SEQ ID NO: 3 and Humulin R (positive control) at concentrations of 0.001, 0.01 and 0.1 μM in a serum-free DMEM medium for 5 min. The cells were washed with a PBS solution, treated with a GLUT4 antibody (Santa Cruz, Calif., USA) for 1 hr, treated with rhodamine red for 40 min, and observed using a confocal laser microscope (Olympus FluoView FW1000; Olympus, Tokyo, Japan). The results are shown in FIG. 7. As is apparent from the results of FIG. 7, the amount of the glucose transporter (GLUT4) was increased in the cell membrane in a concentration-dependent manner, similarly to Humulin R used as the positive control, indicating that the peptide according to the present invention functioned like insulin.

1. A peptide comprising three or four amino acids represented by Chemical Formula 1 below:
X-A-Leu-Y <Chemical Formula 1>
wherein A is serine (Ser) or glutamine (Gln);
X is hydrogen, tyrosine (Tyr) or cysteine (Cys);
Y is hydrogen, tyrosine (Tyr) or glutamic acid (Glu);
neither X nor Y is hydrogen; and
— is a peptide bond.

2. The peptide of claim 1, which is a peptide represented by Chemical Formula 1a below:
X1-Ser-Leu-Y1 <Chemical Formula 1a>
wherein X1 is hydrogen or cysteine (Cys);
Y1 is hydrogen or tyrosine (Tyr);
neither X1 nor Y1 is hydrogen; and
— is a peptide bond.

3. The peptide of claim 2, which is a peptide selected from the group consisting of SEQ ID NOS: 1, 3, and 4.

4. The peptide of claim 2, which is a peptide of SEQ ID NO: 3.

5. The peptide of claim 1, which is a peptide represented by Chemical Formula 1 b below:
X2-Gln-Leu-Y2 <Chemical Formula 1b>
wherein X2 is hydrogen or tyrosine (Tyr);
Y2 is hydrogen or glutamic acid (Glu);
neither X2 nor Y2 is hydrogen; and
— is a peptide bond.

6. The peptide of claim 5, which is a peptide selected from the group consisting of SEQ ID NOS: 2, 5, and 6.

7. A pharmaceutical composition for preventing or treating diabetes or diabetic wounds, containing, as an active ingredient, the peptide comprising three or four amino acids of claim 1.

8. The pharmaceutical composition of claim 7, wherein the peptide is a peptide selected from the group consisting of SEQ ID NOS: 1 to 6.

9. The pharmaceutical composition of claim 7, wherein the peptide is a peptide of SEQ ID NO: 3.

10. The pharmaceutical composition of claim 7, which is provided in a form of a transdermal delivery system.

11. A pharmaceutical composition for preventing or treating diabetes or diabetic wounds, containing, as an active ingredient, the peptide comprising three or four amino acids of claim 2.

12. A pharmaceutical composition for preventing or treating diabetes or diabetic wounds, containing, as an active ingredient, the peptide comprising three or four amino acids of claim 3.

13. A pharmaceutical composition for preventing or treating diabetes or diabetic wounds, containing, as an active ingredient, the peptide comprising three or four amino acids of claim 4.

14. A pharmaceutical composition for preventing or treating diabetes or diabetic wounds, containing, as an active ingredient, the peptide comprising three or four amino acids of claim 5.

15. A pharmaceutical composition for preventing or treating diabetes or diabetic wounds, containing, as an active ingredient, the peptide comprising three or four amino acids of claim 6.
